(19) United States
(12) Patent Application Publication
(10) Pub. No.: US 2013/0256118 A1
(43) Pub. Date: Oct. 3, 2013

(54) Use of Nanopore Arrays For Multiplex Sequencing of Nucleic Acids

(71) Applicants: Amit Meller, Brookline, MA (US); Zhiping Weng, Wellesley, MA (US); Alon Singer, Brighton, MA (US); Benjamin McNally, Brighton, MA (US)

(72) Inventors: Amit Meller, Brookline, MA (US); Zhiping Weng, Wellesley, MA (US); Alon Singer, Brighton, MA (US); Benjamin McNally, Brighton, MA (US)

(73) Assignee: TRUSTEES OF BOSTON UNIVERSITY, Boston, MA (US)

(21) Appl. No.: 13/697,067

(22) PCT Filed: May 11, 2011
(86) PCT No.: PCT/US11/36112

§ 371 Date: Jun. 19, 2013

Related U.S. Application Data
(60) Provisional application No. 61/395,323, filed on May 11, 2010.

(51) Int. Cl. G01N 33/487; C12Q 1/68

(52) U.S. Cl. CPC G01N 33/48721; C12Q 1/6874
USPC 204/158.21; 204/543; 204/519

(57) ABSTRACT

Described are techniques for optical detection of single molecule signals from a nanopore array for analysis of nucleic acid sequences. These techniques are useful for rapid multiplexed DNA sequencing.

### CROSS-REFERENCE TO RELATED APPLICATIONS

This application claims the benefit or priority to U.S. Provisional Application No. 61/395,323, filed May 11, 2010, the entire disclosure of which is hereby incorporated by reference in its entirety.

### GOVERNMENT SUPPORT

This invention was made with Government Support under Contract No. HG-004128 awarded by the National Institutes of Health. The Government has certain rights in the invention.

### FIELD OF THE INVENTION

The present invention relates to the field of nucleic acid analysis. In particular, the invention relates to optical imaging of single molecule signals from a nanopore array for analysis of nucleic acid sequences.

### BACKGROUND

Nanopore-based DNA sequencing is widely considered to be a promising next generation sequencing platform (references [1, 2]). Two main features of the nanopore method make it exceptionally useful for single molecule-based genome analyses: first, the method's ability to electrophoretically focus and thread extremely long DNA molecules from the bulk into the nanopore, making it possible to analyze minute DNA samples (reference [3]) and second, sub-5 nm nanopores are now routinely used to linearize long DNA coils, thus in principle, nanopores can be used to effectively ‘scan’ information along a long genome. These features, as well as the fact that solid-state nanopores can be fabricated in a highly dense array (references [4, 5]), allow the development of massively parallel detection, and are crucial for the realization of an amplification-free, low-cost and high-throughput sequencing (references [2, 6-9]).

A nanopore is a nanometer-sized pore in an ultra-thin membrane that separates two chambers containing an ionic solution. An external electrical field applied across the membrane creates an ionic current and a local electrical potential gradient near the pore, which draws in and threads biopolymers through the pore in a single file manner (references [3, 10]). As a biopolymer enters the pore, it displaces a fraction of the electrolytes, giving rise to a change in the pore conductivity, which can be measured directly using an electrometer. A number of nanopore-based DNA sequencing methods have recently been proposed and highlight two major challenges (as described in references [1, 11]): (1) the ability to discriminate among individual nucleotides, i.e., the system must be capable of differentiating among the four bases on a single-molecule level; and (2) the method must enable parallel readout. As a single nanopore can probe only a single molecule at a time, a strategy for manufacturing an array of nanopores and simultaneously monitoring them is needed.

To date, parallel readout through any nanopore-based method has not yet been demonstrated. A large number of current, and future, generation sequencing methodologies rely on the use of an enzyme (polymerase, exonuclease, etc.) in the readout process. The kinetics of enzymatic activity, however, is a major bottleneck for increasing readout speed, and these methods are at the mercy of enzymes' unperturbed activity.

### SUMMARY OF THE INVENTION

This invention is based, in part, on the discovery that nanopore-based method for high-throughput base recognition is possible without the need for enzymes during the readout stage; and, in part, on the development of a method that enables optical detection of signals from multiple nanopores.

It is understood that any of the embodiments described below can be combined in any desired way unless they are mutually exclusive. It is also understood that any embodiment or combination of embodiments can be applied to each of the aspects described below.

In one aspect, the invention provides a method for analysis of nucleic acids comprising: (a) displacing a plurality of optically-labeled oligonucleotides from a plurality of carrier molecules during controlled translocation of the carrier molecules through a plurality of nanopores in a nanopore array, wherein each carrier molecule passes through a different nanopore in the nanopore array; and (b) detecting a plurality of optical signals from the optically-labeled oligonucleotides as the optically-labeled oligonucleotides are displaced from different carrier molecules.

In some embodiments, the nanopores in a nanopore array are in a solid state membrane with a thickness from about 0.1 nm to about 1 μm.

In some embodiments, the solid state membrane comprises a material which creates a mechanically-stable membrane. In some embodiments, the membrane comprises silicon, silicon nitride, silicon oxide, titanium oxide, aluminum oxide or graphene.

In some embodiments, the nanopores have a diameter of about 1 to about 20 nm. In some embodiments, the nanopores are spaced about 0.5 to about 10 μm apart. In some embodiments, the nanopore array comprises from 2 to about 100,000 nanopores.

In some embodiments, the method further comprises exciting the optical labels associated with the optically-labeled oligonucleotides with a light source. In some embodiments, the light source is a laser.

In some embodiments, the optical labels are excited with a plurality light sources, wherein each light source has a different light emission spectrum. In some embodiments, the optical signals are detected from the surface of the membrane.

In some embodiments, the optical signals are detected with a device capable of recording at least 500 frames per second. In some embodiments, the optical signals are detected with a device capable of recording at least 1,000 frames per second.

In some embodiments, optical detection comprises parallel detection of multiple spectra split onto different regions of an acquisition sensor. In some embodiments, the number of regions is 2. In other embodiments, the number of regions is 4.

In some embodiments, each region on the acquisition sensor produces an individual image per acquired frame. In some embodiments, each image represents a single nucleobase in a nucleic acid sequence of interest.

In some embodiments, the optical signals are detected with a CCD-based camera. In other embodiments, the optical signals are detected with an EM-CCD based camera. In still other embodiments, the optical signals are detected with a CMOS-based camera.

In some embodiments, the optical signals are detected from either side of the membrane. In some embodiments, the optical signals are detected from the cis side of the membrane.

In some embodiments, displacing an optically-labeled oligonucleotide from a carrier molecule passing through a single nanopore in the nanopore array generates a single detectable optical signal.

In some embodiments, the optical signals are fluorescent signals.

In some embodiments, the fluorescent signals from individual nanopores are generated at a rate of at least 500 photon bursts per second.

In some embodiments, each fluorescent signal represents an individual nucleobase in a nucleic acid sequence of interest. In some embodiments, the fluorescence signal allows nucleotide identification based on fluorescence intensity ratios.

In some embodiments, the controlled translocation through a nanopore in the nanopore array is self-regulated via the displacement of discrete optically-labeled oligonucleotides from the carrier molecule.

In some embodiments, the carrier molecule comprises DNA or RNA.

In some embodiments, the method further comprises making the carrier molecule from a nucleic acid sequence of interest by a circular DNA conversion process.

In some embodiments, the carrier molecule is from about 100 to about 50,000 nucleotides in length. In some embodiments, each optically-labeled oligonucleotide represents a single nucleobase in a nucleic acid sequence of interest.

In some embodiments, the controlled translocation through a nanopore in the nanopore array does not utilize an enzyme or a protein.

In some embodiments, from about 100 to about 500 optically-labeled oligonucleotides are displaced per nanopore from a single carrier molecule. In some embodiments, at least 500 optically-labeled oligonucleotides are displaced from a single carrier molecule.

In some embodiments, the self-regulation is based on one or more of the following factors: (i) voltage gradient applied across the membrane; (ii) temperature; (iii) number of nucleobases in the displaced optically-labeled oligonucleotides; (iv) the G-C content of the displaced optically-labeled oligonucleotide; (v) chemical composition of the displaced optically-labeled oligonucleotide; and (vi) electrolyte conditions on either side of the membrane.

In some embodiments, the nanopore diameter controls the rate of capture of carrier molecules.

In some embodiments, the nanopores in the nanopore array are chemically or biologically unmodified.

In some embodiments, the optically-labeled oligonucleotides comprise DNA, RNA, PNA, or LNA.

In some embodiments, the carrier molecule represents the sequence information of a nucleic acid sequence of interest.

In some embodiments, the method further comprises obtaining the sequence of a nucleic acid sequence of interest from sequential detection of optical signals generated by displacement of the optically-labeled oligonucleotides from the carrier molecules.

In some embodiments, the nucleic acid sequence of interest comprises DNA.

In some embodiments, the optical signals from optically-labeled oligonucleotides associated with different carrier molecules are detected simultaneously.

In some embodiments, the method further comprises associating an optical signal with a specific nanopore in the nanopore array.

In another aspect, the invention provides an apparatus for performing the methods described above.

### BRIEF DESCRIPTION OF THE FIGURES

FIG. 1(a) is a schematic illustration of the two steps in the DNA sequencing methodology. First, bulk biochemical conversion of each nucleotide of the target DNA sequence to a known oligonucleotide, followed by hybridization with molecular beacons. Second, threading of the DNA/beacon complex through a nanopore allows optical detection of the target DNA sequence.

FIG. 1(b) is a schematic illustration of the conceptual parallel readout scheme. Each nanopore has a specific location in the visual field of the EM-CCD and therefore enables simultaneous readout of an array of nanopores.

FIG. 2 is a graphic representation of electro/optical detection of bulky group unzipping. (a) Representative events of unzipping 1 bit and 2 bit complexes using sub 5 nm nanopores. Electrical current is at the top, while the optical signal is at the bottom. (b) Histograms (n>600 for each sample) of the total photon counts per event indicate that most complexes in the 1-bit sample (darker grey) produce one photon burst, while most complexes in the 2 bit sample (lighter gray) produce two photon bursts. Solid lines represent Poisson fits to the histograms, with mean values of 1.30±0.06 and 2.65±0.08 for the 1 bit and 2 bits samples, respectively. (c) Classification of events using a single intensity threshold to count the number of photon bursts per event. The 1 bit sample (left three columns) displays ˜90% of the events having 1 photon burst. The 2 bit sample (right three columns) displays ˜80% having 2 photon bursts.

FIG. 3 is a graphic illustration of two-color unzipping experiments with A647 (lighter gray) and A680 (darker gray) fluorophores. (a) Accumulated photon intensity. A single, prominent peak is observed in each channel, indicating nanopore location as imaged on the EM-CCD. The R values, the ratios of fluorescent intensity measured in Channel 1 vs. Channel 2, are 0.2 and 0.4 for the two fluorophores. (b) Electro/optical signals for representative unzipping events with A647 (top) and A680 (bottom). (c) Accumulating hundreds of traces for each sample yielded R=0.20±0.06 and 0.40±0.05 for A647 and A680 respectively. Lines are fits to Gaussian functions.

FIG. 4 is a graphic illustration of optical nanopore nucleotide identification using two fluorophores. (a) 2 colors enable the construction of 2 bit samples which correspond to all four DNA nucleobases. (b) The R distribution generated with >2000 events reveals two modes at 0.21±0.05 and 0.41±0.06, which correspond to the A647 and A680 fluorophores respectively, in excellent agreement with control studies. Line represents a double Gaussian fit function. (c) Representative intensity-corrected fluorescence traces of individual 2-color 2-bit unzipping events, with the corresponding bit called, base called and certainty score indicated above the event. The intensities in the two channels were corrected automatically by a computer code, after each bit is called using a fixed threshold R value. The values in the parenthesis represent certainty value for each base extracted automatically.

FIG. 5 is a graphic illustration of multi-pore detection of DNA unzipping events. (a) Surface plot depicting the accumulated optical intensity clearly depict the locations of three nanopores as imaged by the EM-CCD, fabricated in the SiN membrane. The high-resolution TEM images of the three nanopores (−5 nm each) are shown. (b) Four representative traces display the concurrent unzipping at two different nanopores. Electrical current traces do not contain information on nanopore location, while optical traces (different shades of gray) allow determination of the location of the unzipping event.

### DETAILED DESCRIPTION OF THE INVENTION

In some embodiments, the invention provides an apparatus comprising a state-solid nanopore array, an imaging device capable of optically recording signals from single molecules, and a data recording system for image acquisition and processing for high throughput analysis of optical signals leading to nucleic analysis and specifically low-cost DNA sequencing. The optical signals in some embodiments embodiment are fluorescent signals generated via a light source exciting optically detectable oligonucleotides as they are displaced (unzipped) during translocation of a carrier molecule through a nanopore. Use of the herein-described solid-state nanopore arrays has the benefit of no additional modification by enzymes, proteins, or other chemical substances. Additionally, since the signals are detected optically versus electrically, it is not necessary to connect or address each and every nanopore using electrical circuitry.

### REFERENCES AND DEFINITIONS

The patent and scientific literature referred to herein establishes knowledge that is available to those of skill in the art. The issued U.S. patents, published U.S. applications, published foreign and international applications, and references that are cited herein are hereby incorporated by reference to the same extent as if each was specifically and individually indicated to be incorporated by reference.

As used herein, the “cis side” is the membrane side to which the sample will be initially placed and the “trans” side is the membrane side to which the sample or a portion thereof will be located following translocation through the nanopore.

As used herein, “a mechanically-stable membrane” is a membrane in which an array of nanopores can be produced and is not subject to rupturing when an electrical force is applied across the membrane.

As used herein, “a carrier molecule” is a polymer comprising monomer units to which optically-detectable labeled molecules are capable of binding reversibly. In some embodiments, the carrier molecule comprises from 10-100,000 monomer units. The monomer units are electrically charged, either positive or negative, so as they may be acted upon in an electric field to effect translocation through the solid state nanopore. In some embodiments, carrier molecules are negatively charged strands of single stranded nucleic acid, composed of either RNA or DNA monomers, to which optically-detectable labeled oligonucleotides can be bound (hybridized).

As used herein, “ssDNA” refers to single-stranded DNA and “dsDNA” refers to double-stranded DNA.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous.

As used herein, unless specifically indicated otherwise, the word “or” is used in the inclusive sense of “and/or” and not the exclusive sense of “either/or.”

### Solid State Nanopore Arrays

An array of nanopores or holes is made in a solid-state membrane that is from about 0.1 nm to about 1 um thick. The solid state membrane can be made of silicon, silicon nitride, silicon oxide, titanium oxide, aluminum oxide, or grapheme. In a specific embodiment, the solid state membrane is made of silicon nitride. In some embodiments, the solid state membrane is made of a suitable material which allows the creation of a mechanically-stable membrane. The membrane following formation of a nanopore array is generally stable to the forces applied, i.e., the electrical force used to translocate the carrier molecule, and does not collapse or during the translocation process.

In some embodiments, the nanopores have a diameter ranging from about 1 to about 20 nm, 1-5 nm, 3-5 nm, 2-6 nm, 3-6 nm, or 2-10 nm. The nanopores can be arranged in an ordered array, or may be random in pattern. The spacing of the nanopores can be fixed or variable. In some embodiments, the nanopores are spaced at fixed spacing of from 0.5-10 μm apart, 1-8 μm apart, 2-10 μm apart, 1-2 μm apart or 1.8-7.7 μm apart.

The number of nanopores in a nanopore array can vary. In some embodiments, a nanopore array comprises from 2 to 1,000, from 1,000-10,000, from 10,000-100, or more than 100,000 nanopores. The number of nanopores in a given array is variable depending upon the specific application. In some embodiments, the number of nanopores is such that the field of view of the imaging device is not exceeded thus permitting the device to acquire images of the entire array without moving either the imaging device or the array to acquire additional fields of view.

In some embodiments, solid state nanopore array chips are fabricated starting from a double-sided polished silicon wafer coated with 30 nm thick, low-stress Si₃N₄ (SiN). 30×30 μm² windows exposing both sides of the SiN membrane can be created using wet KOH etching. Nanopores (3-5 nm in diameter) can be fabricated using a focused electron beam, for example, as described in more detail in reference [16]. The drilled nanopore array chips are cleaned and assembled on a custom-designed Teflon cell incorporating a glass coverslip bottom (as described in more detail in reference [13]) under controlled humidity and temperature. Nanopores are hydrated with the addition of degassed and filtered 1M KCl electrolyte to the cis chamber and 1M KCl with 8.6M urea to the trans chamber to facilitate total internal reflection (TIR) imaging. Ag/AgCl electrodes are immersed into each chamber of the cell and connected to an Axon 200B headstage, used to apply a fixed voltage (300 mV for all experiments) across the membrane and to measure the ionic current when needed. Nanopore current can be filtered using a 50 kHz low pass Butterworth filter and sampled using a DAQ board at 250 kHz/16 bit (PCI-6154, National Instruments, TX). Electrical signals can be acquired using a custom LabView program as described in reference [15].

In some embodiments, solid state nanopores are produced as described in U.S. Pat. Nos. 7,258,838, 7,846,738 and published U.S. Pat. App. Nos. 2011-0053284 and 2006-0003458.

In some embodiments, the nanopore arrays are produced in solid-state materials as described in more detail in references 119.201

### Carrier Molecules

In some embodiments, the carrier molecule is a single-stranded nucleic acid which comprises of DNA, RNA, or analogs thereof. DNA and RNA analogs are natural or synthetic variations of the 5 nucleobases (adenine, guanine, cytidine, thymidine, and uracil), the sugar (ribose or 2′-deoxyribose) or phosphate moieties. The carrier molecule may be native genetic material or produced synthetically, for example a circular DNA conversion process. The carrier molecule is hybridized with oligonucleotides that are optically labeled, such that each optically-labeled oligomer is indicative of a single nucleobase or a single target sequence in the DNA of interest.

In some embodiments, the carrier molecule is produced by the process of circular DNA conversion wherein the order (sequence) of each nucleobase of the sample nucleic acid is converted producing a polymer, the monomer units comprise the complementary sequence to one of four corresponding optically-labeled oligomers. The carrier molecule polymer maintains the ordered sequence of nucleobases found in the original sample nucleic acid. Methods for doing so are known in the art and are described, for example, in WO 2010/053820.

In some embodiments, the optically-labeled oligomer(s) hybridize directly to targeted regions, i.e., small sequences of genes, exons, introns, regulatory units, etc, which are indicative of a genetic alteration or a mutation in the sample nucleic acid. In this example the relative position of 2 or more optically-labeled oligomers along the sample nucleic acid carrier molecule is used to identify genetic abnormalities. During the displacement of an optically-labeled oligonucleotide from the carrier molecule, a photon burst is produced from a labeled oligonucleotide which is imaged by the imaging system. Each individual nanopore is able to translocate carrier molecules independent of each and every other nanopore in the array. A given carrier molecule can generate hundreds of displacement events to be recorded during a translocation event. The recorded signals are processed and can be used to generate the nucleic acid sequence information.

### Optically-Labeled Oligonucleotides

In some embodiments, the optically-labeled oligonucleotide is a single-stranded nucleic acid of 10-20, 20-30, or 30-50 nucleotides. In some embodiments, the optically-labeled oligonucleotide comprises DNA, RNA, PNA, LNA or an analog thereof and one or more optical labels attached to it. In some embodiments, the optically-labeled oligonucleotide has a fluorophore and a quencher attached to it. Analogs thereof are natural or synthetic variations of the 5 nucleobases (adenine, guanine, cytidine, thymidine, and uracil), the sugar (ribose or 2′-deoxyribose) or phosphate moieties.

In some embodiments, the optically-labeled oligonucleotide has the configuration of a molecular beacon. Molecular beacons are hairpin shaped molecules with an internally quenched fluorophore, whose fluorescence is restored when they bind to a complementary target nucleic acid sequence. A molecular beacon is an oligonucleotide comprising an overall length of 12-30 nucleotides, a fluorophore on or near one end and a quencher molecule on or near the other end, wherein the ends are able to form an intramolecular double-stranded configuration ranging from 4-10 bases on each end resulting in juxtapositioning the fluorophore and quencher.

### Optical Labels

In some embodiments, lasers are used as the light source to excite fluorescent labels, for example fluorophores in optically-labeled oligonucleotides, which generate the optically-detectable signal. Organic dyes, such as fluorophores, are just one example of the optical labels that can be used. Other examples include inorganic optical labels, such as gold particles or quantum dots. Since fluorophores have optimal wavelengths of excitation, various wavelength lasers each with a different emission spectrum may be used. In some embodiments a single laser may be used to excite multiple fluorophores. In other embodiments, multiple lasers are used to excite multiple fluorophores.

Many fluorophores known in the art are useful as optical labels, for example FAM, TET, HEX, JOE, VIC, Cy2, Cy3, TMR, Oyster dyes, ROX, Texas Red, Cy5, Cy7, IRD700, IRD 770, IRD 800, Alexa and Atto dyes, the only requirement being that when multiple (e.g., 2-4) fluorophores are used each fluorophore is optically resolvable from the other fluorophores used. Any spectral overlap between fluorophores gives rise to some level of uncertainty in its ability to be clearly identified. Quenchers are also widely known in the art, for example Dabcyl, Eclipse, Iowa Black FQ and Iowa Black RQ, BHQ-1, BHQ-2, DDQ-I, DDQ-II, QSY-7 and QSY-21. Some quenchers, for example the BHQ, additionally confer enhance stability of the oligonucleotide hybridization to the carrier increasing the melting temperature approximately 4° C. In some embodiments, the fluorophore-quencher combination is a cyanine fluorophore and a quencher from the BHQ family of quenchers.

In some embodiments, the number of fluorophores able to be individually optically-detected ranges from 1-5 or is more than 5. Each fluorophore must be able to be specifically optically resolvable as a single molecule. In the application of optical detection to DNA sequencing, in some embodiments, the optical signal from one dye corresponds to one nucleobase and a total of four fluorophores are used. However, it is possible to use multiple fluorophores for a single nucleobase. Additionally, in some applications, it might be desirable to detect an additional base such a uracil, so the five nucleobases are adenine (A), guanine (G), cytosine (C), thymidine (T) and uracil (U).

### Detection of Optical Signals

In some embodiments, a high-speed imaging device with a wide field of view capable of viewing the entire array of nanopores is used for optical detection. The imaging device is capable of optically detecting tens of photons from individual fluorophores using total internal reflection (TIR) from the surface of the membrane. The imaging device captures images at a high frame rate, for example, at the rate of at least 500 frames per second. The imaging device sensor is able to split regions of the acquisition sensor wherein each region is corresponds to a different spectral region of light, minimum is two regions. In some embodiments, however, four regions are defined.

Examples of suitable imaging devices include, but are not limited to, a CCD based camera, an CMOS-CCD-based camera and an EM-CCD-based camera. CCD stands for charge-coupled device; CMOS stands for complimentary metal-oxide semiconductor. These are the chips onto which the light captured by a imaging device's lens is focused. Those signals are processed further inside the imaging device and ultimately, the image is recorded to a storage medium, whether its tape, DVD, or an internal or external hard drive. Cameras with multiple image sensors use one chip per light primary color (red, green and blue), and utilize a separate image processor to combine the three signals into a color video image. Newer and cheaper CMOS chips bundle both an image sensor and image processor into a single chip. CCD designs typically utilize an image processor that is separate from the actual light-capturing sensor. EM-CCD is a quantitative digital camera technology that is capable of detecting single photon events whilst maintaining high Quantum Efficiency, achievable by way of a unique electron multiplying structure built into the sensor. Unlike a conventional CCD, an EM-CCD is not limited by the readout noise of the output amplifier, even when operated at high readout speeds. This is achieved by adding a solid state Electron Multiplying (EM) register to the end of the normal serial register; this register allows weak signals to be multiplied before any readout noise is added by the output amplifier, hence rendering the read noise negligible. The EM register has several hundred stages that use higher than normal clock voltages. As charge is transferred through each stage the phenomenon of Impact Ionization is utilized to produce secondary electrons, and hence EM gain. When this is done over several hundred stages, the resultant gain can be (software) controlled from unity to hundreds or even thousands of times.

In some embodiments, the optical imaging system is designed so that lasers and imaging device are located on opposite sides of the membrane with the imaging device on the cis side of the membrane. The cis side of the membrane is the side to which the sample is introduced and resides before passing through the membrane. In an exemplary configuration, the lasers and camera are located on the same side of the nanopore membrane. The cis and trans side are opposite sides of the membrane to which negative and positive polarities are applied, respectively. The orientation of the polarity depends upon the charge on the sample to be translocated.

In some embodiments, the imaging system records signals of photon burst from optically-labeled oligonucleotides as they are displaced (i.e., unzipped) from a carrier molecule during translocation through a single nanopore. In a specific embodiment, the optical labels are fluorophores. The use of fluorophores enables one to couple the spectral signal of a specific fluorophore to a specific nucleobase.

In some embodiments, the optical signal is an intensity wherein an intensity ratio is used to assign signal to a specific nucleobase. Intensity ratios are calculated by summing up the total photon emitted within pre-defined wavelength bands associated with each of the fluorophores used. Then the ratios of each of the bands to the other bands are calculated. This provides a robust way to discriminate between 2 or more emitted colors regardless of their absolute intensities.

In some embodiments, a single laser source can be used to excite two different fluorescent tags. This reduces the number of required laser sources. In such embodiments, there is significant spectral overlap in the emission of the two fluorophores, and therefore there is cross-talk between the two detecting channels. However, by using the relative ratio of the intensities of each fluorophore in both detecting channels, one can differentiate between the two dyes. A benefit of the optical nanopore array as described herein is that many thousands of individual translocations can be recorded in parallel enabling ultra-high throughput sequencing.

In some embodiments, implementation of a four-color detection system that uses one fluorophore for each base halves the converted DNA length (i.e., the length of the carrier molecule) and doubles the detection speed, while also increasing the accuracy for base calling. A four-color system can also remove the error created in the two-color system as a result of potential frame shifting.

Additionally, since the signals are recorded by an optical imaging device there is no requirement for recording electrical signals from each nanopore during the translocation thus enabling the manufacture of low-cost, low-complexity solid state nanopore arrays.

### Regulation of Translocation Through the Nanopores

In some embodiments, the nanopore translocation process can be regulated in order to control the speed at which the carrier molecule translocates through the nanopore. In one embodiment, the design of the optically-labeled oligonucleotides controls the rate of displacement, wherein oligonucleotide length (number of nucleobase units), the G-C content, and or chemical composition (DNA, RNA, PNA, LNA or an analog thereof), is one level of control or self-regulation which determines the speed at which a molecule translocates through a given nanopore. The rate of displacement may also be controlled by external physical controls such as voltage gradient across the membrane, temperature, and electrolyte conditions on either side of the membrane. The nanopore diameter can also be varied to regulate the rate of capture or entry of the carrier molecule into the nanopore. Each of these regulatory elements are independently variable.

The unzipping process of one strand of DNA from another is dictated by an energetic barrier. In some embodiments, the unzipping process is self-regulated and depends on one or more of: (i) voltage gradient applied across the membrane (this increases the force on the molecule and hence reduces the energetic barrier of unzipping); (ii) temperature (temperature can also impact the thermodynamic stability of the duplex and hence alter the energetic barrier of unzipping); (iii) the number of nucleobases in the displaced optically-labeled oligonucleotide dictates the energetic barrier height; (iv) the G-C content of the displaced optically-labeled oligonucleotide dictates the energetic barrier height; (v) the chemical composition of the displaced optically-labeled oligonucleotide (e.g., DNA, RNA, PNA, LNA or an analog thereof; chemically different nucleic acid compositions have differing strength of binding and hence alter the energetic barrier of unzipping; and (vi) electrolyte conditions on either side of the membrane have both an affect on the force applied (in a manner similar to (i), while also impact the screening of charge along the DNA, thus impacting the energetic barrier of unzipping).

In some embodiments, the solid state nanopores do not require modification by chemical or biological molecules in order to be functional. The translocation of the carrier molecule or products thereof is not rate-limited by an enzyme action, thus providing a fast enzyme-free readout.

### Applications for Nucleic Acid Sequencing

Some embodiments of the present invention relate to a method for single molecule, optical imaging on nanopore arrays useful in high-throughput, accurate, low-cost nucleic acid analysis or nucleic acid sequencing. The method is advantageous because it allows the high-throughput analysis of optical (multicolor fluorescent) signals representing the order of individual nucleotides present in either DNA or RNA. The method comprises a low-complexity, solid state membrane comprising an array of drilled nanopores or holes (nanopore array), a light source, an imaging device, and a data recording system capable of image acquisition and processing the signals as optically-detectable labeled oligonucleotides are displaced (unzipped) from a carrier molecule during the controlled translocation through a nanopore.

In some embodiments, the biochemical preparation (circular DNA conversion) of the target DNA molecules converts each nucleobase into a form that can be read directly using an unmodified solid state nanopore. The massively parallel conversion process is performed off-line, and does not require enzyme immobilization or an amplification step. Due to this biochemical preparation step, the nanopore readout speeds and read lengths are not enzyme-limited. Moreover, in contrast to methods using electrical signals to probe biomolecules in nanopores, the methods and apparatus described herein optical detection to detect DNA sequence. A custom Total Internal Reflection (TIR) method, which permits high spatiotemporal resolution and wide-field optical detection of individual DNA molecules translocating through a nanopore, is used for 2-color fast single molecule detection (the TIR method is described in more detail in reference [13]). In some embodiments, the apparatus is able to simultaneously optically detect signals from multiple nanopores.

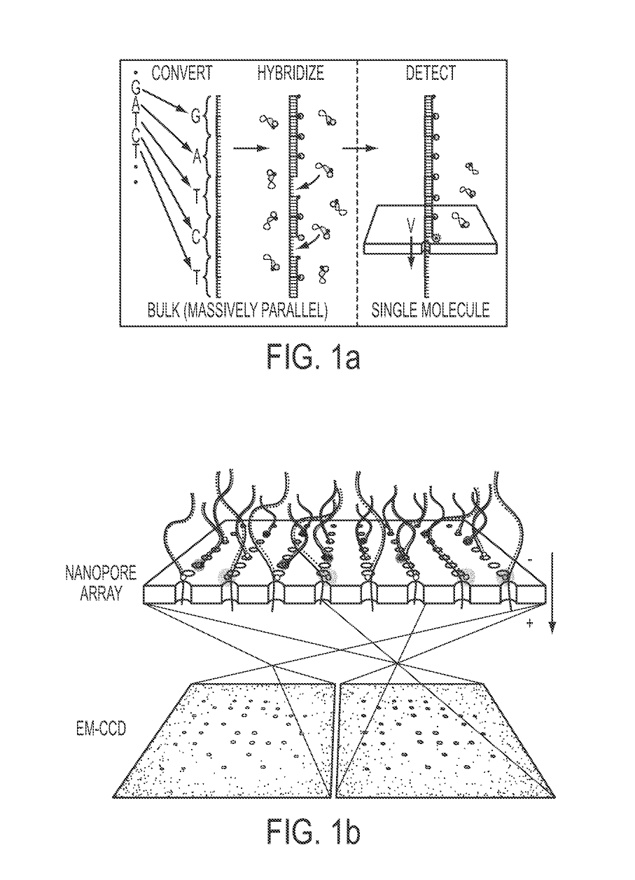

An exemplary approach to DNA sequencing comprises two steps (FIG. 1a): in the first step, each of the four nucleotides (A, C, G and T) in the target DNA is converted to a predefined nucleic acid polymer, which is hybridized with molecular beacons (e.g., optically-labeled oligonucleotides) that carry a specific fluorophore, for example as described in reference [12]. This DNA conversion can be performed, for example, using a method referred to as “Circular DNA Conversion” (CDC). Circular DNA conversion is known in the art and can be done, for example, as described in U.S. Pat. No. 6,723,513. In the second step (FIG. 1a), the hybridized molecule is translocated through a nanopore in a nanopore array and optical signals are detected as the molecular beacons dissociate from the converted DNA molecule (i.e., the carrier molecule).

In embodiments in which two-color readout (i.e., two types of fluorophores) is used, the four sequences are combinations of two predefined unique sequences, bit ‘0’ and bit ‘1’, such that an A would be ‘11’, a G would be ‘1 0’, a T would be ‘01’ and finally a C would be ‘00’ (FIG. 1a). Two types of molecular beacons, carrying two types of fluorophores, hybridize specifically to the ‘0’ and ‘1’ sequences. The converted DNA and hybridized molecular beacons are electrophoretically threaded through a solid-state nanopore, where the optically-labeled oligonucleotides, also referred to as beacons, are sequentially stripped off (displaced or unzipped). Each time a beacon is stripped off, a new fluorophore is unquenched, giving rise to a burst of photons, recorded at the location of the nanopore (FIG. 1b). The sequence of two-color photon bursts, at each nanopore location, (shown in FIG. 1b) is the binary code of the target DNA sequence. This approach circumvents the need to detect individual bases and facilitates an enzyme-free readout. Additionally, this method permits wide-field imaging and spatially fixed nanopores enable straightforward adaptation to simultaneous detection of multiple nanopores with a electron multiplying charge coupled device (EM-CCD) camera (schematically illustrated in FIG. 1b).

In some embodiments, the readout uses a solid-state nanopore to strip hybridized molecular beacons off converted single-strand DNA molecules (carrier molecules). This generally requires the use of pores in the sub-2 nm range, because the cross-section diameter of double-stranded DNA (dsDNA) is 2.2 nm. However, the probability of DNA entry into such small pores is much smaller than larger pores, necessitating the use of a larger amount of DNA. Moreover, routine manufacturing of small pores poses technical challenges, as there is little tolerance for error, and the difficulty escalates for high-density nanopore arrays. It has been discovered that covalently attaching a 3-5 nm sized “bulky” group (e.g., a protein or a nanoparticle) to the molecular beacons effectively increases the molecular cross-section of the complex to 5-7 nm, allowing the use of nanopores in the size range of 3-6 nm. This increases the capture rate of DNA molecules by 10-fold or more, and greatly facilitates the fabrication process of the nanopore arrays.

In some embodiments, custom TIR imaging is used to achieve high-speed single molecule detection of individual fluorophores near the suspended silicon nitride membrane. The index of refraction of the trans chamber solution can be adjusted, such that TIR can be created at the SiN membrane, preventing light from progressing into the cis chamber and thus reducing additional background. The cell can be mounted on a high NA objective (Olympus 60X/1.45), and TIR can be optimized by focusing the incident 640 nm laser beam (iFlex2000, Point-Source UK) to an off-axis point at its back focal plane, thereby controlling the angle of incidence. Fluorescence emission can be split into two separate optical paths using a dichroic mirror (e.g., Semrock, FF685Di01) and the two images can be projected side by side onto an EM-CCD camera (e.g., Andor, iXon DU-860). The EM-CCD worked at maximum gain and 1 ms integration time. Synchronization between the electrical and optical signals can be achieved by connecting the camera ‘fire’ pulse to a counter board (e.g., PCI-6602, National Instruments), which shared the same sampling clock and start trigger as the main DAQ board. The combined data stream can include unique time stamps at the beginning of each CCD frame, which were synched with the ion current sampling. Two separate criteria can be used for classifying each event. The first criterion can be an abrupt drop of the ion current below a user defined threshold level, and remain at that level for at least 100 us, before returning to the original state. The second criterion can be an increase in the photon count only at the region of the nanopore during the event dwell-time (time where signal stays below the threshold) in the corresponding CCD frames.

This invention is further illustrated by the following example, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### EXAMPLES

### Example 1

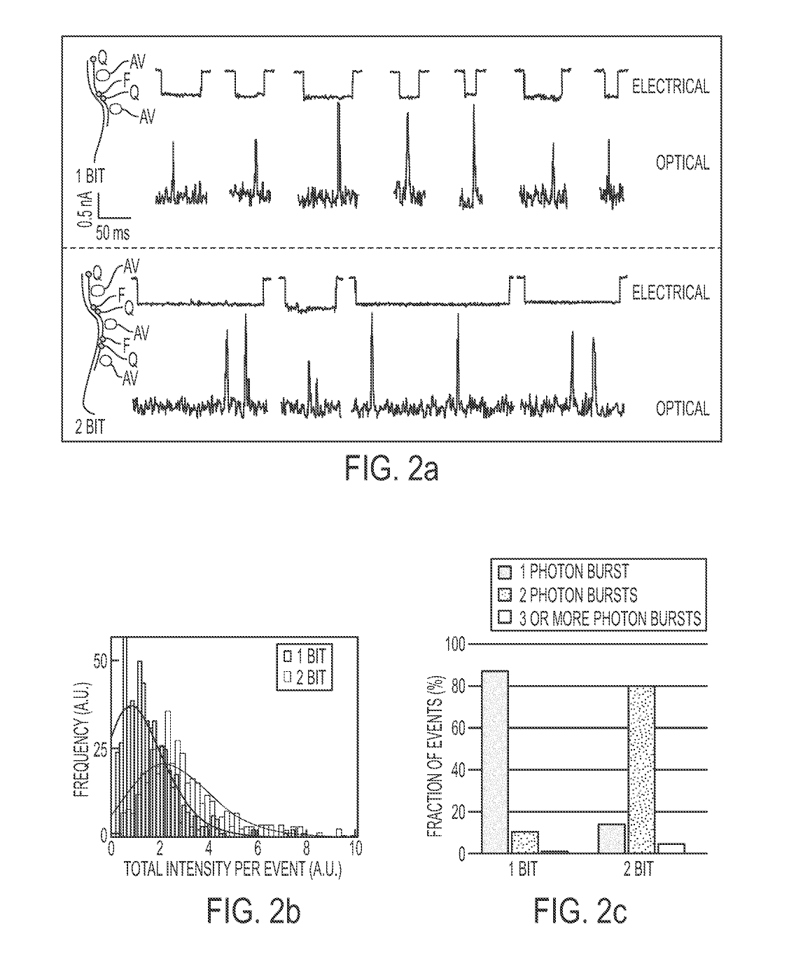

Avidin (4.0×5.5×6.0 nm) was bound to a biotinylated molecular beacon containing a fluorophore-quencher pair (ATTO647N-BHQ2, abbreviated as “A647-BHQ”). Both this beacon and a similarly constructed oligonucleotide, containing a quencher at one end but no fluorophore at the other end, were hybridized to a target ssDNA (‘1 bit’ sample). A similar complex was synthesized containing two beacon molecules (‘2 bit’ sample), as shown schematically in FIG. 2a. Bulk studies demonstrated that, when in its hybridized state, the A647 fluorophore is quenched ˜95% by the neighboring BHQ quencher. Given this extremely high quenching efficiency, fluorescence bursts can be detected at the single-molecule level only if strand separation occurs.

Nanopore experiments for both the 1-bit sample (containing 1 beacon molecule percomplex) and the 2-bit sample (containing 2 beacon molecules per complex) were carried out using a 640 nm laser and imaged at 1,000 frames per second using an EM-CCD camera. FIG. 2a displays typical unzipping events for the two samples, with one beacon per complex in the 1-bit sample, and two beacons per complex in the 2-bit sample. Electrical signals are shown in black, and optical signals, measured synchronously with the electrical signals at the nanopore position, in different shades of gray. An abrupt decrease in electrical current signifies the entry of the molecule to the pore, and the pore is cleared when the electrical signal returns to the open-pore upper state. It is noted that the unzipping events observed here are substantially longer than previously reported (reference [13]) due to the presence of the bulky group. The optical signals clearly show either one or two photon bursts for the 1-bit and 2-bit samples, respectively. This is expected since the fluorophores are quenched before reaching the pore and are self-quenched again immediately after the beacons are unzipped from the template. A simple summation of the optical intensity during each event, as defined by the electrical signal, yields Poisson distributions for the two samples (solid lines in FIG. 2b), with mean values of 1.30±0.06 for the 1-bit sample, and double 2.65±0.08 for the 2-bit sample (n>600 events in each case, errors represent STD). This proves that regardless of a model used to define a photon burst, a single unzipping event occurred for the 1-bit sample and two unzipping events occurred for the 2-bit samples. Moreover, with the use of an intensity threshold analysis (average intensity+2 STD) it was determined that nearly 90% of the collected events in the 1 bit sample contained a single fluorescent burst, while in the 2 bit sample, ˜80% of the collected events displayed 2 such bursts (FIG. 2c). This data demonstrates that it is possible to optically discriminate between 1 bit and 2 bit samples, in individual unzipping events performed using a 3-5 nm nanopore.

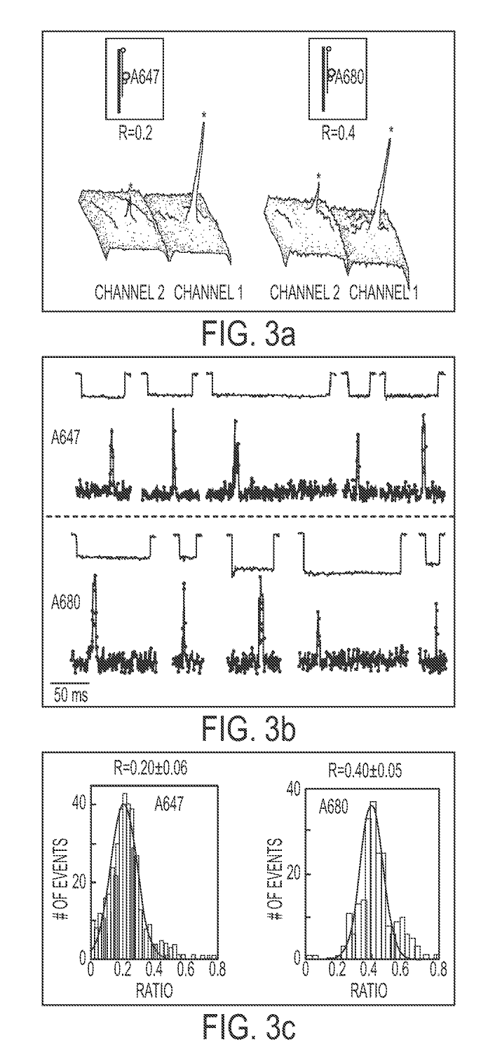

To distinguish between all four nucleotides, the system was extended from a 1 color to a 2 color coding scheme using two high quantum yield fluorophores, A647 (ATTO647N) and A680 (ATTO680), excited simultaneously by the same 640 nm laser. The optical emission signal was split into channels 1 and 2 using a dichroic mirror and imaged side-by-side on the same EM-CCD camera. Two-color intensity analysis was performed by reading the intensity at a 3×3 pixel area centered at the nanopore position (see for example FIG. 3a). As the emission spectra of the two fluorophores overlap, a fraction of the A647 emission “leaks” into channel 2, and a fraction of A680 “leaks” to channel 1. Two calibration measurements were performed using 1-bit complexes labeled with A647 or A680 fluorophores (FIG. 3a). Clearly seen is a single distinct peak in each channel, corresponding to the location of the nanopore, after accumulation of >500 unzipping events in each case. The ratio of the fluorescent intensities in Channel 2 vs. Channel 1 (R) is 0.2 for the A647 sample, and 0.4 for the A680 sample.

Representative events (out of more than 500) for each for the two samples, and the corresponding distributions of R, are depicted in FIGS. 3b and 3c, respectively. A single prominent fluorescent peak is observed during each translocation event (electrical traces shown in black), with intensity >3 fold larger than the standard deviation of the fluorescence baseline fluctuations. Tallying up all single-molecule events led to R=0.20±0.06 and 0.40±0.05 (mean±std) for A647 and A680, respectively, in complete agreement with the ratios for accumulated fluorescence (for all events) shown in FIG. 3a. R follows a Gaussian distribution, given by the solid line fits in FIG. 3c. These control measurements show that it is possible to use R to determine the identity of individual fluorophores. Discrimination was performed automatically in a custom LabView code, using the calibration data (FIG. 3c). The error in the determination of each of the two dyes can be calculated from the overlap area between the distributions, yielding <9% for the A647 and <13% for A680. Data analysis was performed using IGOR Pro (Wavemetrics), and fits were created to optimize chi-square.

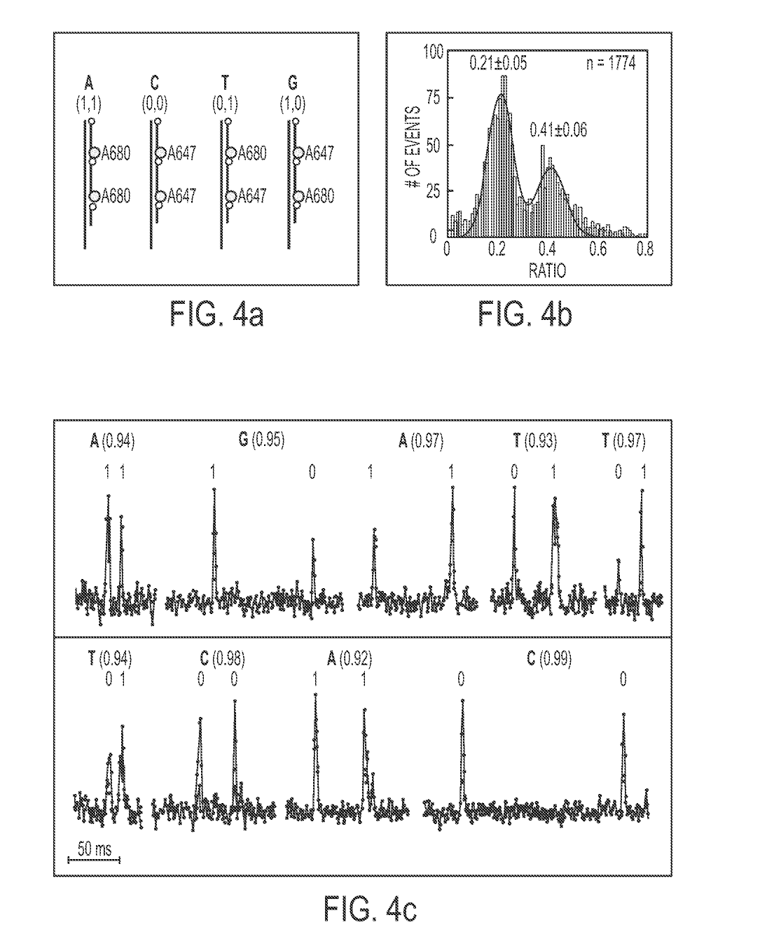

Using the calibration distributions given in FIG. 3c, the ability to identify the products from the circular DNA conversion containing the four 2-bit combinations for all four bases, namely 11 (A), 00 (C), 01 (T), and 10 (G), where “0” and “1” correspond to the A647 and A680 beacons, respectively, was tested. Analysis of >2000 unzipping events, in which 2 distinct photon bursts were detected, revealed a bimodal distribution of R, with two modes at 0.21±0.05 and 0.41±0.06 (FIG. 4b), in complete agreement with the calibration measurements (FIG. 3c). All photon bursts with R<0.30 were classified as “0”, and those with R>0.30 as “1” (0.30 is the local minimum of the distribution in FIG. 4b). The distribution of R was also used to compute the probability of misclassification. This provides us with a further statistical means to calibrate the two channels for optimal discrimination between the two fluorophores. FIG. 4c presents representative 2-color fluorescence intensity events depicting the single molecule identification of all 4 DNA bases.

The robustness of the two-color identification is attributed primarily to the excellent signal/background levels of the photon bursts and the separation between the fluorophore intensity ratios for the two channels. A computer algorithm was developed to perform automatic peak identification that also filters out random noise (e.g., false spikes) in the fluorescence signals and identifies the bit sequence using the calibration distributions (FIG. 3c), followed by base calling. The algorithm outputs two certainty scores, one for bit calling and the other one for base calling. Typical results are shown in FIG. 4c. The certainty value for each base extracted automatically from the raw intensity data (range between 0 and 1) is displayed in parenthesis.

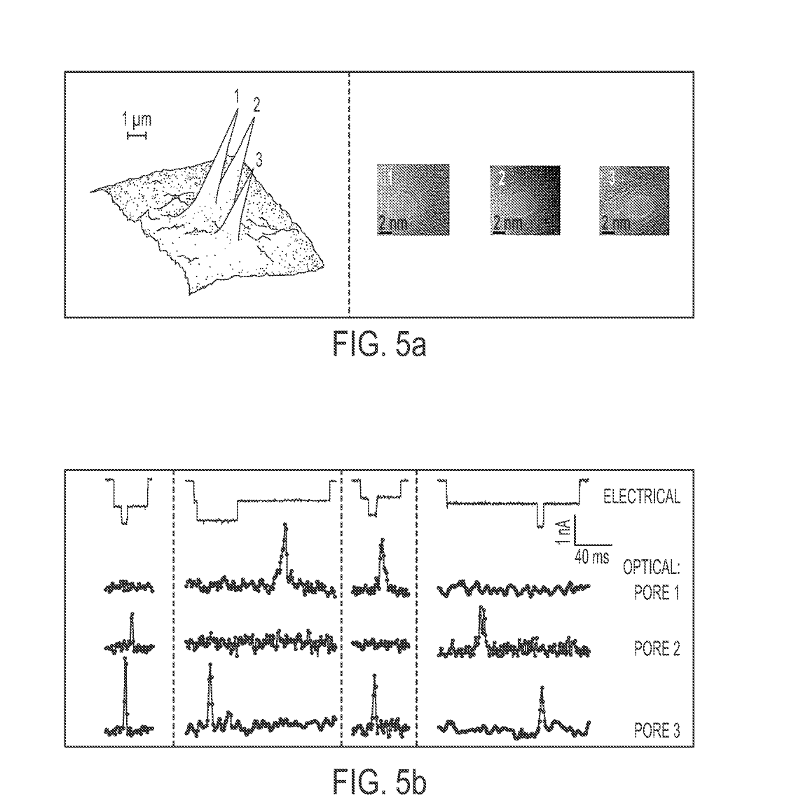

One of the advantages of a wide-field optical-based detection scheme lies in the simplicity with which multiple nanopores can be probed in parallel, ultimately enabling high-throughput readout. As a proof of concept for parallel readout, multiple 3-5 nm sized nanopores were fabricated on the same silicon nitride membranes, separated by several microns. In FIG. 5a, accumulated fluorescence intensity images are displayed, obtained using membranes containing three nanopores. Like the single nanopore experiments, fluorescent bursts were recorded from all nanopores in the membrane. Accumulating photon counts from several thousand unzipping events resulted in surface maps of photon intensity at each pixel. The distance between the three peaks for the three-pore membrane were 1.8 μm and 7.7 μm, in agreement with the distances between the nanopores measured during the fabrication process. This data provides direct evidence for the feasibility of a wide-field optical detection scheme.

In FIG. 5b, the ability of the system to probe photon bursts simultaneously from multiple nanopores in a single membrane was demonstrate. Four representative traces show the electrical current and the optical signal using 1-bit sample probed from the three nanopores. The entrance and unzipping process of each molecule, at each nanopore, is a stochastic process. It was found that under the conditions used in this experiment, out of >3,000 unzipping events, ˜50 involved molecules entering through two nanopores at the same time. The electrical current trace, which is accumulated from all nanopores, displays two distinct blockade levels, indicating the total number of occupied nanopores at a particular moment, without information on which nanopores are occupied. The optical traces reveal occupied nanopores unambiguously. This will ultimately eliminate the need for electrical current measurements when the methods described herein are extended to larger arrays, and rely solely on optical measurements, simplifying instrumentation requirements.

DNA sequencing methods using nanopores offer several advantages over alternative methods. The speed of readout is completely controllable by adjusting the applied voltage, and is only limited by the detection modality resolution. Future developments of brighter fluorophores and higher-sensitivity CCDs can straightforwardly translate to faster readout speeds. As a single-molecule method, it does not have large sample concentration stipulations and therefore aids in driving down both cost and sample amplification error. Finally, the nanopore readout shown here does not involve the immobilization of enzymes onto predefined or random locations, thus highly simplifying the reading platform. Here the feasibility of two color converted DNA readout using a binary code (2 bits per base) to represent each DNA base was demonstrated. In some embodiments, the system can read 50-250 bases per second per nanopore.

It is expected that a straightforward adaptation for 4-color and the use of optimized reagents will allow achieving at least 500 bases per second, per nanopore and sharply decrease the base classification errors. Even when of-the-shelf reagents were used and a single laser line was employed, the nucleotide classification error is about 10% (per single read). Since the DNA conversion process produces a structure-free DNA it automatically removes systematic errors from the readout stage (i.e., errors do not depend on the DNA template sequence). Therefore the predominant source of readout error can be substantially lowered with multiple reads of the same sequence. Lastly, the feasibility of multi-pore readout was demonstrated, which is believed to be a first for nanopore based methods.

The results described herein results indicate the feasibility of using solid-state nanopores for optical DNA sequencing.

The results demonstrate what is believed to be the first all-solid-state DNA sequence readout. Such an ultra-fast and affordable system has numerous applications in biomedical research and in the diagnosis and treatment of human diseases.

### REFERENCES

1. Branton, D., D. W. Deamer, A. Marziali, H. Bayley, S. A. Benner, T. Butler, M. Di Ventra, S. Garaj, A. Hibbs, X. H. Huang, S. B. Jovanovich, P. S. Krstic, S. Lindsay, X. S. S. Ling, C. H. Mastrangelo, A. Meller, J. S. Oliver, Y. V. Pershin, J. M. Ramsey, R. Riehn, G. V. Soni, V. Tabard-Cossa, M. Wanunu, M. Wiggin, and J. A. Schloss. 2008. The potential and challenges of nanopore sequencing. Nat Biotechnol 26:1146-1153.
2. Healy, K. 2007. Nanopore-based single-molecule DNA analysis. Nanomedicine 2:459-481.
3. Wanunu, M., W. Morrison, Y. Rabin, A. Y. Grosberg, and A. Meller. 2010. Electrostatic focusing of unlabelled DNA into nanoscale pores using a salt gradient. Nature Nanotech. 5:160-165.
4. Nam, S.-W., M. J. Rooks, K.-B. Kim, and S. M. Rossnagel. 2009. Ionic Field Effect Transistors with Sub-10 nm Multiple Nanopores. Nano Lett 9:2044-2048.
5. Lee, H.-S., B.-S. Kim, H.-M. Kim, J.-S. Wi, S.-W. Nam, K.-B. Jin, Y. Arai, and K.-B. Kim. 2007. Electron Beam Projection Nanopatterning Using Crystal Lattice Images Obtained from High Resolution Transmission Electron Microscopy. Adv Mater 19:4189-4193.
6. Harris, T. D., P. R. Buzby, H. Babcock, E. Beer, J. Bowers, I. Braslaysky, M. Causey, J. Colonell, J. DiMeo, J. W. Efcavitch, E. Giladi, J. Gill, J. Healy, M. Jarosz, D. Lapen, K. Moulton, S. R. Quake, K. Steinmann, E. Thayer, A. Tyurina, R. Ward, H. Weiss, and Z. Xie. 2008. Single-Molecule DNA Sequencing of a Viral Genome. Science 320:106-109.
7. Shendure, J., R. D. Mitra, C. Varma, and G. M. Church. 2004. Advanced sequencing technologies: Methods and goals. Nat Rev Genet 5:335-344.
8. Eid, J., A. Fehr, J. Gray, K. Luong, J. Lyle, G. Otto, P. Peluso, D. Rank, P. Baybayan, B. Bettman, A. Bibillo, K. Bjornson, B. Chaudhuri, F. Christians, R. Cicero, S. Clark, R. Dalal, A. Dewinter, J. Dixon, M. Foquet, A. Gaertner, P. Hardenbol, C. Heiner, K. Hester, D. Holden, G. Kearns, X. X. Kong, R. Kuse, Y. Lacroix, S. Lin, P. Lundquist, C. C. Ma, P. Marks, M. Maxham, D. Murphy, I. Park, T. Pham, M. Phillips, J. Roy, R. Sebra, G. Shen, J. Sorenson, A. Tomaney, K. Travers, M. Trulson, J. Vieceli, J. Wegener, D. Wu, A. Yang, D. Zaccarin, P. Zhao, F. Zhong, J. Korlach, and S. Turner. 2009. Real-Time DNA Sequencing from Single Polymerase Molecules. Science 323:133-138.
9. Fuller, C. W., L. R. Middendorf, S. A. Benner, G. M. Church, T. Harris, X. Huang, S. B. Jovanovich, J. R. Nelson, J. A. Schloss, D. C. Schwartz, and D. V. Vezenov. 2009. The challenges of sequencing by synthesis. Nat Biotech 27:1013-1023.
10. Deamer, D. W., and D. Branton. 2002. Characterization of Nucleic Acids by Nanopore Analysis. Accounts Chem Res 35:817-825.
11. Clarke, J., H. C. Wu, L. Jayasinghe, A. Patel, S. Reid, and H. Bayley. 2009. Continuous base identification for single-molecule nanopore DNA sequencing. Nature Nanotech. 4:265-270.
12. Soni, G., and A. Meller. 2007. Progress towards ultra-fast DNA sequencing using solid-state nanopores. Clin. Chem. 53:1996-2001.
13. Soni, G. V., A. Singer, Z. Yu, Y. Sun, B. McNally, and A. Meller. 2010. Synchronous optical and electrical detection of biomolecules traversing through solid-state nanopores. Rev. Sci. Instrum. 81:014301-014307.
14. McNally, B., M. Wanunu, and A. Meller. 2008. Electromechanical Unzipping of Individual DNA Molecules Using Synthetic Sub-2 nm Pores. Nano Lett 8:3418-3422.
15. Wanunu, M., J. Sutin, B. McNally, A. Chow, and A. Meller. 2008. DNA Translocation Governed by Interactions with Solid-State Nanopores. Biophys J 95:4716-4725.
16. Kim, M. J., M. Wanunu, D. C. Bell, and A. Meller. 2006. Rapid fabrication of uniformly sized nanopores and nanopore arrays for parallel DNA analysis. Adv Mater 18:3149-3153.
17. Green, N. M., and M. A. Joynson. 1970. A preliminary crystallographic investigation of avidin. Biochem J 118:71-72.
18. Bonnet, G. g., O. Krichevsky, and A. Libchaber. 1998. Kinetics of conformational fluctuations in DNA hairpin-loops. P Natl Acad Sci USA 95:8602-8606.
19. Tong, H. D., H. V. Jansen, V. J. Gadgil, C. G. Bostan, E. Berenschot, C. J. M. van Rijn, and M. Elwenspoek. 2004. Silicon Nitride Nanosieve Membrane. Nano Lett 4:283-287.
20. Wico, C. L. H., and et al. 2007. Focused ion beam scan routine, dwell time and dose optimizations for submicrometre period planar photonic crystal components and stamps in silicon. Nanotechnology 18:195305.

1. A method for analysis of nucleic acids comprising:
(a) displacing a plurality of optically-labeled oligonucleotides from a plurality of carrier molecules during controlled translocation of the carrier molecules through a plurality of nanopores in a nanopore array, wherein each carrier molecule passes through a different nanopore in the nanopore array; and
(b) detecting a plurality of optical signals from the optically-labeled oligonucleotides as the optically-labeled oligonucleotides are displaced from different carrier molecules.

2. The method of claim 1, wherein the nanopores in the nanopore array are in a solid state membrane with a thickness from about 0.1 nm to about 1 μm.

3. The method of claim 2, wherein the solid-state membrane comprises a material which creates a mechanically-stable membrane.

4. The method of claim 1, wherein the membrane comprises silicon, silicon nitride, silicon oxide, titanium oxide, aluminum oxide or graphene.

5. The method of claim 1, wherein the nanopores have a diameter of about 1 to about 20 nm.

6-7. (canceled)

8. The method of claim 1, further comprising exciting the optical labels associated with the optically-labeled oligonucleotides with a light source.

9. (canceled)

10. The method of claim 8, wherein the optical labels are excited with a plurality of light sources, wherein each light source has a different light emission spectrum.

11. The method of claim 2, wherein the optical signals are detected from the surface of the membrane.

12-13. (canceled)

14. The method of claim 1, wherein optical detection comprises parallel detection of multiple spectra split onto different regions of an acquisition sensor.

15. (canceled)

16. (canceled)

17-21. (canceled)

22. The method of claim 1, wherein the optical signals are detected from either side of the membrane.

23. (canceled)

24. The method of claim 1, wherein displacing an optically-labeled oligonucleotide from a carrier molecule passing through a single nanopore in the nanopore array generates a single detectable optical signal.

25. The method of claim 1, wherein the optical signals are fluorescent signals.

26. (canceled)

27. The method of claim 25, wherein each fluorescent signal represents an individual nucleobase in a nucleic acid sequence of interest.

28. (canceled)

29. The method of claim 27, wherein the controlled translocation through a nanopore in the nanopore array is self-regulated via the displacement of discrete optically-labeled oligonucleotides from the carrier molecule.

30. The method of claim 1, wherein the carrier molecule comprises DNA or RNA.

31-33. (canceled)

34. The method of claim 1, wherein the controlled translocation through a nanopore in the nanopore array does not utilize an enzyme or a protein.

35-36. (canceled)

37. The method of claim 29, wherein the self-regulation is based on one or more of the following factors: (i) voltage gradient applied across the membrane; (ii) temperature; (iii) number of nucleobases in the displaced optically-labeled oligonucleotides; (iv) the G-C content of the displaced optically-labeled oligonucleotide; (v) chemical composition of the displaced optically-labeled oligonucleotide; and (vi) electrolyte conditions on either side of the membrane.

38. (canceled)

39. The method of claim 1, wherein the nanopores in the nanopore array are chemically or biologically unmodified.

40. The method of claim 1, wherein the optically-labeled oligonucleotides comprise DNA, RNA, PNA, or LNA.

41. (canceled)

42. The method of claim 1, further comprising obtaining the sequence of a nucleic acid sequence of interest from sequential detection of optical signals generated by displacement of the optically-labeled oligonucleotides from the carrier molecules.

43-46. (canceled)
